(19) United States
(12) Patent Application Publication
(10) Pub. No.: US 2017/0200898 A1
(43) Pub. Date: Jul. 13, 2017

(54) CARBON NANOTUBE ORGANIC SEMICONDUCTOR, MANUFACTURING METHOD THEREOF, AND TRANSISTOR FOR CHEMICAL SENSOR USING THE SAME

(71) Applicant: DONGGUK UNIVERSITY INDUSTRAY-ACADEMIC COOPERATION FOUNDATION, Seoul (KR)

(72) Inventor: Yong Young NOH, Daejeon (KR)

(73) Assignee: DONGGUK UNIVERSITY INDUSTRAY-ACADEMIC COOPERATION FOUNDATION, Seoul (KR)

(21) Appl. No.: 15/315,388

(22) PCT Filed: Jun. 26, 2015
(86) PCT No.: PCT/KR2015/006559

§ 371 Date: Dec. 1, 2016

(30) Foreign Application Priority Data
Jun. 27, 2014 (KR) 10-2014-0079922

(51) Int. Cl. H01L 51/00; G01N 33/497; G01N 33/574

(52) U.S. Cl. CPC H01L 51/0048; H01L 51/0039; G01N 33/57423; G01N 33/497; H01L 51/0541

(57) ABSTRACT

The present invention relates to a carbon nanotube organic semiconductor, a manufacturing method thereof, and a thin film transistor for chemical sensor using the same. More specifically, the present invention provides a carbon nanotube organic semiconductor, a manufacturing method thereof, and a thin film transistor for chemical sensor using the same, where the carbon nanotube organic semiconductor is an organic semiconductor layer constituting an organic thin film transistor and comprising a conjugated polymer and a single-walled carbon nanotube, the single-walled carbon nanotube displaying semiconducting properties and being wrapped with the conjugated polymer.

### TECHNICAL FIELD

The present invention relates to a carbon nanotube organic semiconductor, a thin film transistor comprising the same, and a chemical sensor and an application using the same. More particularly, the present invention relates to a carbon nanotube organic semiconductor with enhanced performance of electronic devices, a thin film transistor comprising the carbon nanotube organic semiconductor, and a chemical sensor serving as a sensor for gas or the like using the thin film transistor.

### BACKGROUND ART

Human's exhaled breath contains various volatile organic compounds (VOCs). The concentration of VOCs in the exhaled breath of a patient with lung cancer, around 10 to 100 ppb, is relatively high in relation to that in the exhaled breath of a healthy person that is 1 to 20 ppb. For that reason, many studies have recently been made on the techniques for the diagnosis of lung cancer through the analysis of VOCs in the exhaled breath of a patient using this mechanism rather than a surgical operation or a complicated examination. Yet, for the analysis of VOCs, it is necessary to use expensive and complicated analysis equipment with high-sensitivity sensors but low analysis speed, like gas chromatography/mass spectrometry, ion flow tube mass spectrometry, laser absorption spectrometry, infrared spectroscopy, polymer-coated surface acoustic wave sensors, coated quartz crystal microbalance sensors, etc. Accordingly, a variety of chemical sensors have recently been studied in order to cope with the method using such complex, but slow and expensive analysis equipment.

In recent years, there have been many studies actively made on the techniques to realize a chemical sensor using a carbon nanotube (CNT) or organic semiconductor as an active layer of transistors or diodes to detect the change of electrical properties upon exposure to certain chemical substances. The carbon nanotube (CNT) or organic semiconductor, in particular, enables the manufacture of a semiconductor layer using the solution process and has many advantages in the aspect of the production cost of sensors. But, the carbon nanotube (CNT), synthesized to have both metallic and semiconducting properties, has the difficulty in separating the metallic and semiconducting properties and thus results in the limitation in its commercial application.

In the prior art, no more than one semiconductor material such as a carbon nanotube (CNT) or an organic semiconductor is used as an active layer to fabricate a sensor, which results in the limitation to the type of the chemical substance detectable with one semiconductor material. It is therefore impossible to detect various chemical substances in a simultaneous manner with a single device. Moreover, the carbon nanotube (CNT) is sensitive to all the different chemical substances, making it difficult to selectively detect the chemical substances. It is therefore demanded to provide a chemical sensor for the diagnosis of lung cancer from the exhaled breath that uses the mixture of at least two semiconductor materials in a solution as an active layer of a transistor formed by the printing process, realizes the transistor as a device for sensor to perform a simultaneous detection of multiple chemical substances and a selective detection of a specific chemical substance with enhanced sensitivity, and accurately diagnoses lung cancer from the exhaled breath of patients using the device for sensor.

### PRIOR ART DOCUMENTS

International Laid-Open Patent Publication WO 2008-090969

KR Laid-Open Patent Publication No. 2009-0080653

### DISCLOSURE OF INVENTION

It is an object of the present invention to provide a flexible sensor circuit comprising a chemical sensor, and a sensor for detecting the concentration of an organic compound contained in the exhaled breath of a person using the flexible sensor circuit.

It is another object of the present invention to provide a sensor technique for the diagnosis of lung cancer and a system for monitoring the sensed signals with a smartphone application (mobile app).

### Technical Solution

In order to achieve the objects of the present invention, there is provided a carbon nanotube organic semiconductor comprising an organic semiconductor layer constituting an organic thin film transistor, where the organic semiconductor layer comprising a conjugated polymer and a single-walled carbon nanotube. The single-walled carbon nanotube has semiconducting properties and is selectively wrapped with the conjugated polymer.

The conjugated polymer of the present invention is polyfluorene polymer.

The carbon nanotube organic semiconductor of the present invention comprises 0.0001 to 0.015 mg/ml of the single-walled carbon nanotube.

The organic semiconductor layer of the present invention is mixed with a second organic semiconductor. The second organic semiconductor is an N type semiconductor or a P type semiconductor.

At a mixed volume of the carbon nanotube wrapped with the conjugated polymer and the second organic semiconductor, the carbon nanotube wrapped with the conjugated polymer is 10 vol. % or greater in volume.

The N type organic semiconductor of the present invention is selected from a substance based on acene, fully fluorinated acene, partially fluorinated acene, partially fluorinated oligothiophene, fullerene, fullerne with a substituent, fully fluorinated phthalocyanine, partially fluorinated phthalocyanine, perylene tetracarboxylic diimide, perylene tetracarboxylic dianhydride, naphthalene tetracarboxylic diimide, or naphthalene tetracarboxylic dianhydride, or a derivative thereof. The P type organic semiconductor of the present invention is selected from a substance including acene, poly-thienylene vinylene, poly-3-hexylthiophene, alpha-hexathienylene, naphthalene, alpha-6-thiophene, alpha-4-thiophene, rubrene, polythiophene, polyparaphenylene vinylene, polyparaphenylene, polyfluorene, polythiophene vinylene, polythiophene-heterocyclic aromatic copolymer, or triaryl amine, or a derivative thereof.

The present invention also provides a method for manufacturing a carbon nanotube organic semiconductor, which is a layer constituting an organic thin film transistor, the method comprising: (1) mixing a conjugated polymer and a single-walled carbon nanotube in a solvent; (2) performing an ultrasonication on the mixed solution; (3) performing a separation using a centrifugal separator to collet a supernate; and (4) using the supernate to form a carbon nanotube organic semiconductor forming an organic semiconductor layer, where the supernate in the separation step comprises a single-walled carbon nanotube having semiconducting properties wrapped with a conjugated polymer.

The mixing step (1) of the present invention uses 4 to 6 mg of the conjugated polymer and 1.5 to 3.0 mg of the single-walled carbon nanotube per 1 ml of the solvent, and the mixing ratio of the conjugated polymer to the single-walled carbon nanotube is 3:2 to 3:1.

The conjugated polymer of the present invention is any one selected from the group consisting of polyfluorene, polythiophene, 1,4-diketopyrrolo[3,4-c]pyrrole (DPP), naphthalene diimide, naphthalene-bis(dicarboximide) (NDI), isoindigo, and isothiophene indigo.

The solvent of the present invention is any one selected from the group consisting of toluene, chloroform, chlorobenzene, dichlorobenzene, trichlorobenzene, and xylene.

The present invention also provides a transistor for chemical sensor comprising: a substrate; source/drain electrodes disposed apart from each other on the substrate; a carbon nanotube organic semiconductor layer comprising a substance formed of a single-walled carbon nanotube having semiconducting properties wrapped with a conjugated polymer and being disposed on the whole surface of the substrate including the source/drain electrodes; a gate insulating layer being disposed on the whole surface of the organic semiconductor layer; and a gate electrode being disposed on the gate insulating layer.

The conjugated polymer of the carbon nanotube organic semiconductor is any one selected from the group consisting of polyfluorene, polythiophene, 1,4-diketopyrrolo[3,4-c]pyrrole (DPP), naphthalene diimide, naphthalene-bis(dicarboximide) (NDI), isoindigo, and isothiophene indigo.

The carbon nanotube organic semiconductor of the present invention comprises 0.0001 to 0.015 mg/ml of the single-walled carbon nanotube.

The organic semiconductor layer of the present invention uses a second organic semiconductor further added, and the second organic semiconductor is an N type organic semiconductor or a P type organic semiconductor.

At a mixed volume of the carbon nanotube wrapped with the conjugated polymer and the second organic semiconductor, the carbon nanotube wrapped with the conjugated polymer is 10 vol. % or greater in volume.

The transistor of the present invention is used as an active layer to sense the change of chemical properties upon exposure to a chemical substance and applicable to diagnosis of lung cancer with exhaled breath.

### Advantageous Effects

The carbon nanotube organic semiconductor, the thin film transistor comprising the carbon nanotube organic semiconductor, and a chemical sensor using the thin film transistor according to the present invention have an effect to provide a flexible sensor circuit and a sensor for sensing the concentration of organic compounds contained in the exhaled breath of a person.

The carbon nanotube organic semiconductor, the thin film transistor comprising the carbon nanotube organic semiconductor, and a chemical sensor and application using the thin film transistor according to the present invention have an effect to provide a sensor technique for the diagnosis of lung cancer and a system for monitoring the sensed signals with a smartphone application (mobile app).

The carbon nanotube organic semiconductor, the thin film transistor comprising the carbon nanotube organic semiconductor, and a chemical sensor using the thin film transistor according to the present invention can realize a portable chemical sensor that is easily bent or flexed due to the flexibility of its material. The chemical sensor is also applicable as a wearable sensor that can be attached to the body or clothing.

The carbon nanotube organic semiconductor, the thin film transistor comprising the carbon nanotube organic semiconductor, and a chemical sensor using the thin film transistor according to the present invention enable the use of the printing process and reduce the production cost of the conventional sensors to enhance the price competitiveness. With such a high price competitiveness, the present invention can provide a flexible sensor for detecting diseases such as lung cancer simply by detecting various organic compounds contained in the exhaled breath of a patient.

### BRIEF DESCRIPTION OF DRAWINGS

FIG. 1 is a diagram showing the process for manufacturing a thin film transistor according to one embodiment of the present invention.

FIG. 2 is a diagram showing the process for manufacturing a carbon nanotube organic semiconductor according to one embodiment of the present invention.

FIG. 3 is a schematic diagram showing a carbon nanotube wrapped with a conjugated polymer.

FIG. 4 shows UV-vis spectra of the carbon nanotube dispersed in the supernate.

FIG. 5 shows the height image of the thin film forming the carbon nanotube semiconductor layer.

FIG. 6 presents the transition curves of a transistor according to one embodiment of the present invention.

FIG. 7 shows the resistance as a function of the time in the case of injection of ammonia in Example 1 of the present invention.

FIG. 8 shows the resistance as a function of the time in the case of injection of ammonia in Comparative Example 1.

### BEST MODES FOR CARRYING OUT THE PRESENT INVENTION

Hereinafter, the present invention will be described in detail with reference to the accompanying drawings. Reference should be made to the drawings, in which the same reference numerals are used throughout the different drawings to designate the same or similar components as possible. Further, in the following description of the present invention, a detailed description of known configurations and functions incorporated herein will be omitted when it may make the subject matter of the present invention rather unclear.

The term “about or approximately” or “substantially” used in this specification are intended to have meanings close to numerical values or ranges specified with an allowable error and to prevent accurate or absolute numerical values disclosed for understanding of the present invention from being illegally or unfairly used by any unconscionable third party.

The thin film transistor of the present invention may be a transistor of a carbon nanotube organic semiconductor complex. In the present invention, the transistor is described in association with the top gate bottom contact (TGBC) structure, yet it is also applicable to the bottom gate top contact (BGTC) structure as well.

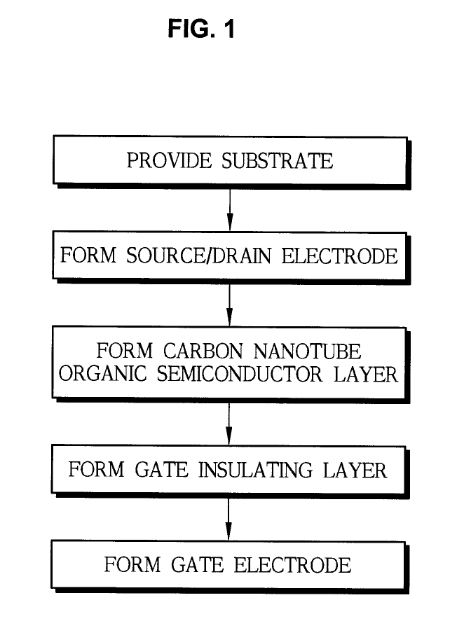

FIG. 1 is a diagram showing the process for manufacturing a chemical sensor using a carbon nanotube organic semiconductor complex in accordance with one embodiment of the present invention.

The present invention provides a transistor of the carbon nanotube organic semiconductor and a chemical sensor using the transistor. The top gate type organic thin film transistor is manufactured in the steps of preparing a substrate; forming source/drain electrodes to be disposed apart from each other on the substrate; forming an organic semiconductor layer to cover the source/drain electrodes; forming an organic semiconductor layer on the carbon nanotube organic semiconductor; forming a gate insulating layer on the organic semiconductor layer; and forming a gate electrode on a partial region of the gate insulating layer.

Referring to FIG. 1, a substrate is provided, and source/drain electrodes are formed on the substrate so that they are disposed apart from each other.

Examples of the substrate may include, but are not limited to, an N type or P type silicon wafer, a glass substrate, a plastic film selected from the group consisting of polyether sulphone, polyacrylate, polyether imide, polyimide, polyethylene terephthalate, and polyethylene naphthalate, or a glass substrate or plastic film coated with indium tin oxide.

The source/drain electrodes may be formed as a single layer selected from Au, Al, Ag, Mg, Ca, Yb, Cs-ITO, or alloy thereof; or as a multi-layer that further includes an adhesive metal layer like Ti, Cr or Ni in order to enhance the adhesion to the substrate. Moreover, graphene, carbon nanotube (CNT), PEDOT:PSS conductive polymer, silver nanowire, etc. can be used to manufacture a flexible device having much higher elasticity than the existing metals. These substances can also be used as an ink for the printing process like ink-jet printing or spraying to make source/drain electrodes. Using the printing process to form source/drain electrodes enables it to exclude the vacuuming process, ending up reducing the production cost.

A carbon nanotube organic semiconductor may be formed on the whole surface of the substrate including the source/drain electrodes.

The carbon nanotube organic semiconductor may be formed with a conjugated polymer wrapping the carbon nanotubes.

The carbon nanotube organic semiconductor may include 0.0001 to 0.015 mg/ml of single-walled carbon nanotubes contained in the conjugated polymer.

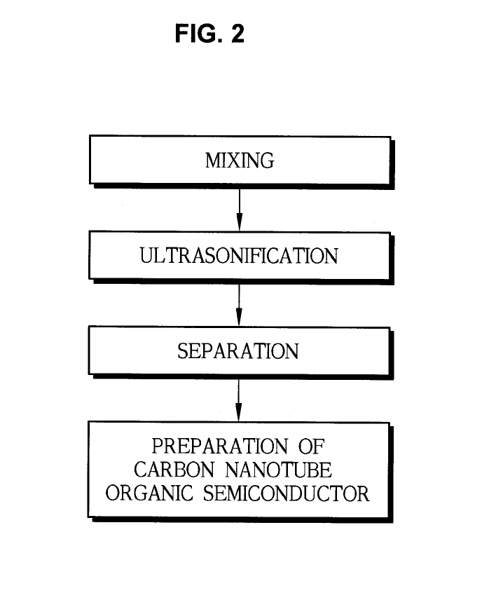

FIG. 2 is a diagram showing the process for manufacturing a carbon nanotube organic semiconductor layer according to one embodiment of the present invention.

The method for manufacturing a carbon nanotube organic semiconductor layer comprises: mixing a conjugated polymer and a single-walled carbon nanotube in a solvent; performing an ultrasonication on the mixed solution; performing a separation using a centrifugal separator to collect a supernate; and using the supernate to form a carbon nanotube organic semiconductor layer.

First of all, the mixing step may involve mixing a conjugated polymer and a single-walled carbon nanotube in a solvent. Preferably, the mixing step includes using 4 to 6 mg of the conjugated polymer and 1.5 to 3.0 mg of the single-walled carbon nanotube per 1 ml of the solvent. The mixing ratio of the conjugated polymer to the single-walled carbon nanotube is preferably in the range of 3:2 to 3:1.

The defined range of the mixing ratio secures the single-walled carbon nanotube and the conjugated polymer well dispersed and mixed together in the solvent.

Examples of the solvent as used herein may include toluene, chloroform, chlorobenzene, dichlorobenzene, trichlorobenzene, xylene, etc.

Preferably, the conjugated polymer of the present invention is polyfluorene (poly[9,9-dioctylfluorenyl-2,7-diyl] (PFO)). The polyfluorene imparts dispersion force to the carbon nanotube and helps forming a complex of carbon nanotube and polyfluorene so that the carbon nanotube is wrapped with the conjugated polymer, polyfluorene.

Beside polyfluorene, other examples of the conjugated polymer as used herein may include any one selected from the group consisting of polythiophene, 1,4-diketopyrrolo[3,4-c]pyrrole (DPP), naphthalene diimide, naphthalene-bis(dicarboximide) (NDI), isoindigo, and isothiophene indigo.

The single-walled carbon nanotube, particularly wrapped with polyfluorene (PFO) used as a conjugated polymer, is not only soluble in a solvent to form an organic semiconductor layer by the inkjet printing or the like, but also applicable to a chemical sensor using the combination of the conjugated polymer and the carbon nanotube to sense the reaction of sensitive gases.

The mixed solution is subjected to ultrasonification, which may be carried out at 15 to 50 Hz for about 30 to 60 minutes.

The ultrasonification on the mixed solution ends up forming a structure having the semiconducting single-walled carbon nanotube wrapped with the conjugated polymer.

The single-walled carbon nanotube displays two characteristics: semiconducting and metallic properties. The present invention selectively makes the use of the semiconducting SWNT. The substance under ultrasonification forms a structure having the single-walled carbon nanotube wrapped with the conjugated polymer. At this point, only the single-walled carbon nanotubes having the semiconducting properties can be wrapped with the conjugated polymer.

On the other hand, the organic semiconductor layer may comprise the carbon nanotube wrapped with the conjugated polymer alone or in combination with another organic semiconductor material.

The second organic semiconductor material may be an N type organic semiconductor or a P type organic semiconductor. Examples of the N type organic semiconductor as used herein may be selected from the substances based on acene, fully fluorinated acene, partially fluorinated acene, partially fluorinated oligothiophene, fullerene, fullerne with a substituent, fully fluorinated phthalocyanine, partially fluorinated phthalocyanine, perylene tetracarboxylic diimide, perylene tetracarboxylic dianhydride, naphthalene tetracarboxylic diimide, or naphthalene tetracarboxylic dianhydride; or derivatives thereof.

Further, examples of the P type organic semiconductor as used herein may be selected from a substance including acene, poly-thienylene vinylene, poly-3-hexylthiophene, alpha-hexathienylene, naphthalene, alpha-6-thiophene, alpha-4-thiophene, rubrene, polythiophene, polyparaphenylene vinylene, polyparaphenylene, polyfluorene, polythiophene vinylene, polythiophene-heterocyclic aromatic copolymer, or triaryl amine; or a derivative thereof.

When used in combination with another organic semiconductor material, the carbon nanotube wrapped with the conjugated polymer is preferably used in an amount of 10 vol. % or greater.

As the carbon nanotube wrapped with the conjugated polymer makes up at least 10 vol. % of the total volume of the semiconductor layer, it is available as a chemical sensor capable of detecting a low concentration of gas.

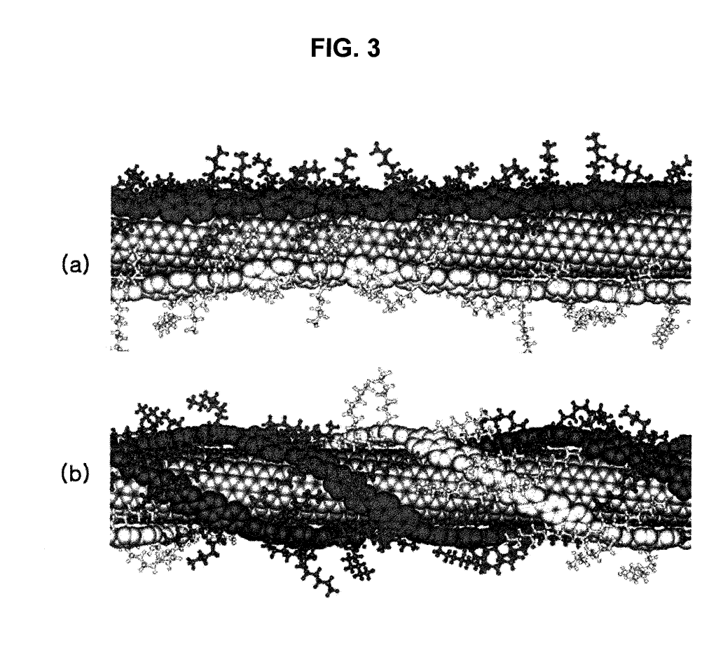

FIG. 3 is a schematic diagram showing a carbon nanotube wrapped with a conjugated polymer.

The conjugated polymer surrounds the single-walled carbon nanotube in such a way that the conjugated polymer molecules are arranged in parallel as shown in FIG. 3(a) or in a twisted form as shown in FIG. 3(b).

The carbon nanotube wrapped with the conjugated polymer has a lower specific gravity than that without the conjugated polymer, so it can be isolated in the separation step.

The separation step uses a centrifugal separator to have the wrapped carbon nanotube get suspended, so that the supernate is collected to separate the wrapped carbon nanotube out.

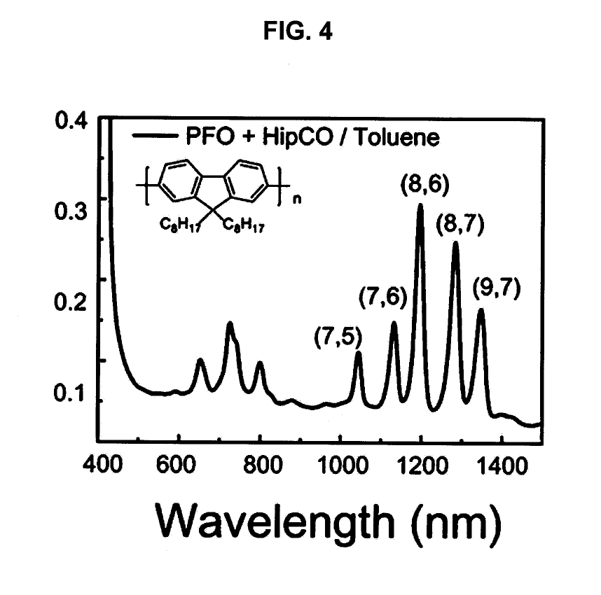

It is observed that the single-walled carbon nanotube dispersed in the supernate is the wrapped carbon nanotube having semiconducting properties. FIG. 4 shows UV-vis spectra of the carbon nanotube dispersed in the supernate.

The single-walled carbon nanotube dispersed in the supernate is proved to be a semiconducting single-walled carbon nanotube. FIG. 4 presents UV-vis spectra of the carbon nanotube dispersed in the supernate using PFO as the conjugated polymer.

In the UV-vis spectra, the semiconducting single-walled carbon nanotube absorbs light in the wavelength range of 1,000 to 1,400 nm, whereas the metallic single-walled carbon nanotube absorbs light in the range of 500 to 600 nm.

Referring to the UV-vis spectra of FIG. 4, peaks appear in the wavelength range of 1,000 to 1400 nm rather than 500 to 600 nm. This shows that the supernate contains a semiconducting single-walled carbon nanotube.

That is, the absorption spectra of the toluene solution containing polyfluorene and the carbon nanotube together show that only the semiconducting carbon nanotubes having chirality of (7,5), (7,6), (8,6), (8,7) and (9,7) are separated. Using this, the polyfluorene (PFO) is selectively bound to the surface of the chiral semiconductor CNT through the pi attraction, so a semiconductor-wrapped carbon nanotube complex can be made in a solvent such as toluene.

The centrifugal separation is preferably performed with a weight of 8,000 to 10,000 g. The supernate obtained by the centrifugal separation is collected and used as an interlayer between the source/drain electrodes and the semiconductor layer. That is, the supernate may be used to form an organic semiconductor layer.

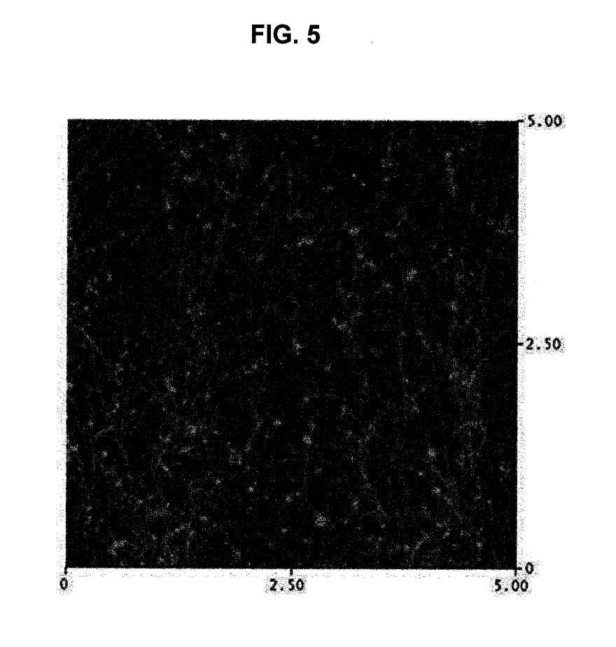

FIG. 5 shows the height image of the thin film forming the carbon nanotube semiconductor layer. Referring to FIG. 5, the single-walled carbon nanotubes are dispersed in the thin film comprised of an organic semiconductor layer.

As the carbon nanotube organic semiconductor is formed on the whole surface of the source/drain electrodes, the trap decreases to increase the charge mobility, ending up enhancing the electronic devices.

On the whole surface of the semiconductor layer may be formed a gate insulating layer.

The gate insulating layer may comprise a single layer or a multi-layer of an organic or inorganic insulating layer; or an organic-inorganic hybrid layer. The organic insulating layer uses at least one selected from the group consisting of polymethylmethacrylate (PMMA), polystyrene (PS), phenol-based polymer, acryl-based polymer, imide-based polymer such as polyimide, acrylether-based polymer, amide-based polymer, fluorine-based polymer, p-xylene-based polymer, vinylalcohol-based polymer, and perylene. The inorganic insulating layer uses at least one selected from the group consisting of silicon oxide, silicon nitride, Al₂O₃, Ta₂O₅, BST, and PZT.

On a part of the region of the gate insulating layer may be formed a gate electrode. The gate electrode may comprise any one selected from the group consisting of aluminum (Al), Al-alloy, molybdenium (Mo), Mo-alloy, silver nanowire, gallium indium eutectic, and PEDOT:PSS. The gate electrode may be prepared through the printing process, such as ink-jet printing or spraying, using the above-mentioned substances as ink. Using the printing process to form the gate electrode can exclude the vacuuming process and thus reduce the production cost.

In this manner, the thin film transistor according to the present invention is completed.

The present invention may also provide a chemical sensor using the thin film transistor.

In the principle that works to operate the chemical sensor, the transistor of the present invention operated by the current amount difference of transistors is used to measure a predetermined current flowing through the channel at a specific gate and source voltage. At this point, the current amount decreases or increases as a gas or chemical substance detectable flows through the transistor.

This enables the sensor to sense the presence of the gas or measure the concentration of the gas according to the increment of the current amount that depends on the concentration of the gas.

According to the present invention, such a detection of gas can be used for the diagnosis of lung cancer. That is, the concentration of the volatile organic compounds (VOCs) in the exhaled breath of a patient is measured to diagnose lung cancer.

The present invention provides a chemical sensor available to diagnose lung cancer in exhaled breath using the thin film transistor as an active layer to sense the change of the electrical properties upon exposure to chemical substances.

The exhaled breath of patients with lung cancer contains volatile organic compounds (VOCs) at higher concentration than that of healthy persons. For example, the concentration of ammonia is 20 to 100 ppm (parts per billion) in the exhaled breath of patients with lung cancer, and 0 to 10 ppb in the exhaled breath of healthy persons. The chemical sensor can detect this to diagnose lung cancer.

In addition, lung cancer can also be diagnosed by detecting the concentration of isopropanol, acetone, or ethanol in the exhaled breath.

In the case of patients with lung cancer, the exhaled breath contains 230 to 1,000 ppb of isopropanol, 150 to 900 ppb of acetone, or 60 to 2,100 ppb of ethanol. This can be detected with the chemical sensor to simply diagnose lung cancer.

The present invention can provide the manufacture of the chemical sensor and the use of a smartphone application (mobile app) to diagnose lung cancer using the chemical sensor.

The chemical sensor is used to construct an active matrix sensor for detecting signals by the increase of the current. The detected signals are changed into voltage signals through the capacitance. The intensity of the output voltage signal varies depending on the concentration of the gas. This enables the determination of the exact concentration of a target chemical substance. The signal of the active matrix sensor is changed into a digital signal through an analog-digital converter, and the output signal is finally sent to a Bluetooth chip by wireless. The Bluetooth chip sends the signal to the paired nearby smart phone to display the exact concentration of a specific chemical substance by way of the installed smartphone application (mobile app).

This system of the present invention measures the concentration of ammonia or other various volatile organic compounds (VOCs) in the exhaled breath blown to the sensor and diagnoses lung cancer according to whether the detected concentration of ammonia is higher than the concentration of ammonia in the exhaled breath of a healthy person. Accordingly, the present invention is applicable to a portable diagnosis system for lung cancer. In particular, the sensor and the electronic circuitry other than the Bluetooth chip can be made on a flexible substrate by various printing processes to remarkably reduce the production cost of the sensor and realize an inexpensive disposable flexible sensor system with good price competitiveness.

### MODES FOR CARRYING OUT THE INVENTION

Hereinafter, a detailed description will be given as to Example and Comparative Example.

### Preparation of Carbon Nanotube Organic Semiconductor

Toluene is prepared as a solvent. And, a single-walled carbon nanotube and polyfluorene (PFO) used as a conjugated polymer are also provided.

4 mg of PFO and 2 mg of a single-walled carbon nanotube are mixed together in 1 ml of toluene, in the mixing step. The mixed solution is then subjected to ultrasonification; that is, it is put into an ultrasonification bath at 20 Hz for 30 minutes and then a tip sonicator for 15 minutes, in the ultrasonification step.

Upon completion of the ultrasonification process, the resulting substance is subjected to centrifugal separation using a centrifugal separator. At this point, the centrifugal separation is carried out for 5 minutes with a centrifugal separator using a weight of 9,000 g. The supernate thus obtained is collected and used in the preparation of a carbon nanotube organic semiconductor.

### Preparation of Thin Film Transistor

The manufacture of a thin film transistor involves the steps of forming source/drain electrodes to be disposed apart from each other on the substrate; forming a carbon nanotube organic semiconductor to cover the source/drain electrodes; forming an organic semiconductor layer on the carbon nanotube organic semiconductor; forming a gate insulating layer on the organic semiconductor layer; and forming a gate electrode on a partial region of the gate insulating layer.

In this regard, the substrate is a glass substrate, and the source/drain electrodes are formed on the substrate through the printing process. On the source/drain electrodes is disposed the carbon nanotube organic semiconductor that is prepared according to the “Preparation of Carbon Nanotube Organic Semiconductor”.

The gate insulating layer is formed from PMMA, and the gate electrode is formed from aluminum (Al) to complete a thin film transistor.

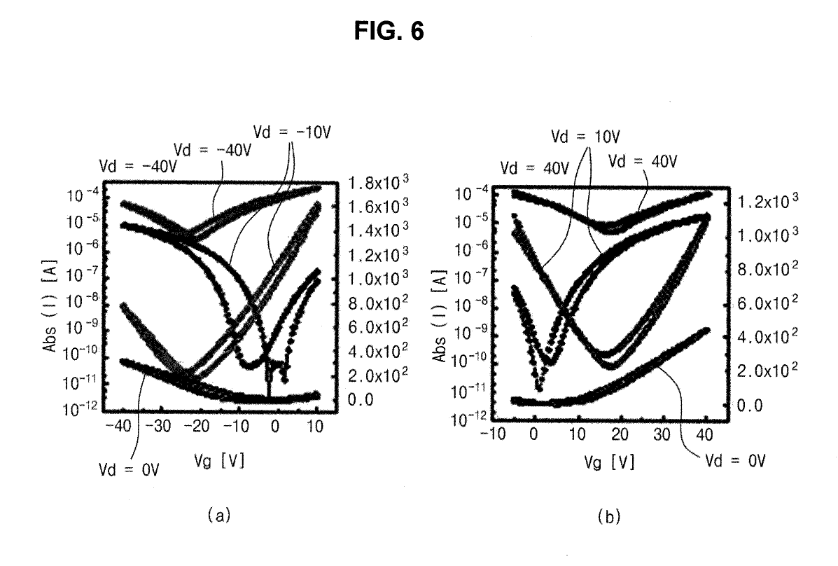

FIG. 6 presents the transition curves of a transistor manufactured in Example 1 of the present invention.

Referring to FIG. 6, a semiconducting carbon nanotube wrapped with a polyfluorene conjugated polymer is applied into a thin film by the spin coating process (500 rpm, 1 min) to complete a transistor. The transistor thus manufactured displays amphiphillic charge properties. At this point, the electron mobility is 1.5 cm²/Vs, the hole mobility is 2.0 cm²/Vs, and the on/off rate is 10⁶ or greater. As this transistor has the sufficiently higher on/off ratio than the existing carbon nanotube (CNT) having both conducting and semiconducting properties, it can be used to manufacture a chemical sensor with high performance.

### Qualification of Chemical Sensor

### Example 1

A resistance type chemical sensor is fabricated based on the above-described carbon nanotube organic semiconductor. More specifically, a single-walled carbon nanotube wrapped with a conjugated polymer (polyfluorene (PFO)) is applied on the surface of a substrate by spin coating, and two electrodes are formed using Au. Then, a voltage of 1 V is applied to measure the resistance value.

To perform a test for determining the sensitivity of the chemical sensor, the chemical sensor is exposed to 10 ppm of gaseous ammonia (NH₃) in a closed space.

The test is performed for 70 minutes. The chemical sensor is exposed to gaseous ammonia (NH₃) in 5 minutes and the supply of the gaseous ammonia (NH₃) is cut off in 25 minutes.

### Comparative Example 1

A pure conducting carbon nanotube (CNT) destitute of a conjugated polymer is applied on the surface of a substrate by spin coating, and two electrodes are formed using Au. Then, a voltage of 1 V is applied to measure the resistance value.

To perform a test for determining the sensitivity of the chemical sensor, the chemical sensor is exposed to 10 ppm of gaseous ammonia (NH₃) in a closed space in the same manner as described in Example 1.

The test is performed for 70 minutes. The chemical sensor is exposed to gaseous ammonia (NH₃) in 5 minutes and the supply of the gaseous ammonia (NH₃) is cut off in 25 minutes.

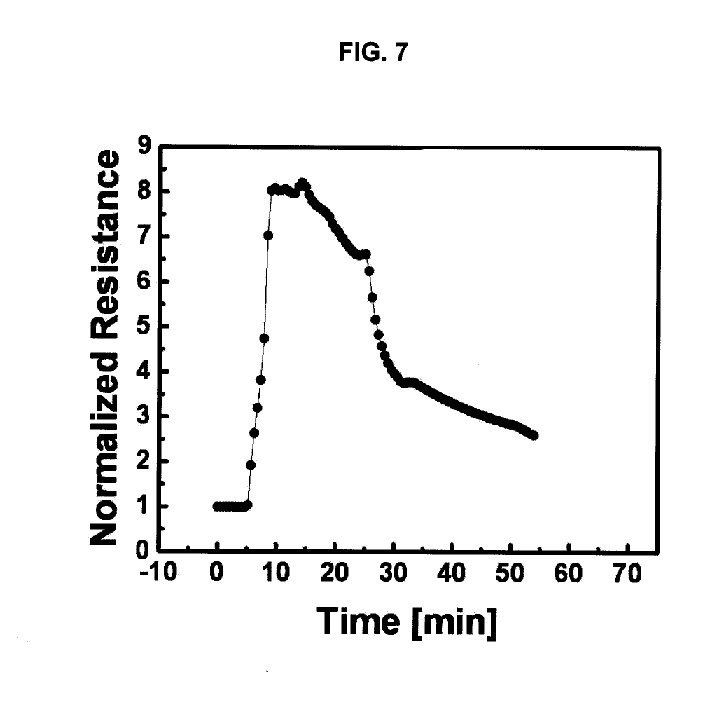

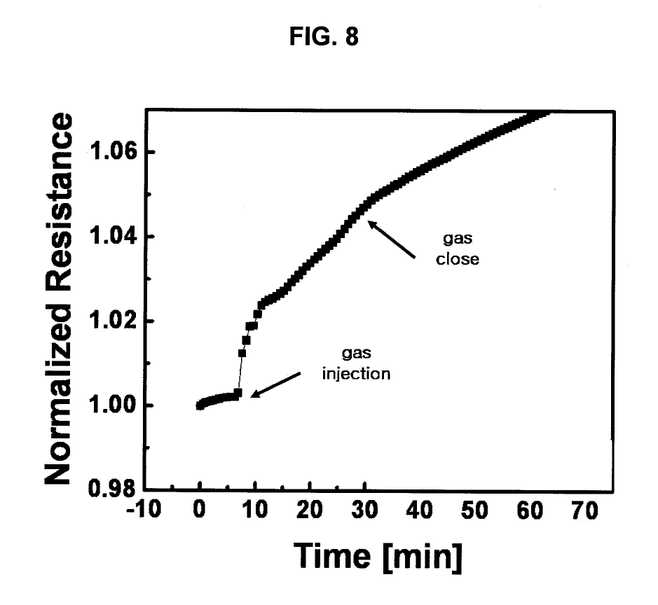

FIGS. 7 and 8 show the change of the resistance value as a function of the time in the case of injection of ammonia in Example 1 and Comparative Example 1, respectively.

It can be seen from the figures that Example 1 of the present invention is about 800 times higher in the change of resistance than Comparative Example 1. This shows that the present invention displays a considerably high sensitivity to ammonia. Accordingly, the electronic devices fabricated from the present invention are available as chemical sensors.

### Chemical Sensor and Application

The thin film transistor fabricated above is used to sense the current change upon exposure to the exhaled breath of a person. The current is decreased or increased when the transistor is exposed to the exhaled breath, which change is detected to determine the concentration of ammonia.

That is, the thin film transistor is used as an active layer for a chemical sensor for diagnosing lung cancer from the exhaled breath.

The present invention may also use a smartphone application (mobile app) to diagnose lung cancer using the chemical sensor. The chemical sensor may be connected to an active matrix sensor, so that the signal of the sensor is sent to the Bluetooth chip by wireless. Further, the smartphone application (mobile app) may be used with a program for the diagnosis of lung cancer to detect the signals from the Bluetooth chip and diagnose lung cancer.

The foregoing description of the invention has been presented for purposes of illustration and description and is not intended to be exhaustive or to limit the invention to the precise form disclosed, and obviously many modifications and variations are possible in light of the above teaching.

1. A carbon nanotube organic semiconductor comprising an organic semiconductor layer constituting an organic thin film transistor,
the organic semiconductor layer comprising a conjugated polymer and a single-walled carbon nanotube,
wherein the single-walled carbon nanotube has semiconducting properties and is selectively wrapped with the conjugated polymer.

2. The carbon nanotube organic semiconductor as claimed in claim 1, wherein the conjugated polymer is any one selected from the group consisting of polyfluorene, polythiophene, 1,4-diketopyrrolo[3,4-c]pyrrole (DPP), naphthalene diimide, naphthalene-bis(dicarboximide) (NDI), isoindigo, and isothiophene indigo.

3. The carbon nanotube organic semiconductor as claimed in claim 1, wherein the carbon nanotube organic semiconductor comprises 0.0001 to 0.015 mg/ml of the single-walled carbon nanotube.

4. The carbon nanotube organic semiconductor as claimed in claim 1, wherein the organic semiconductor layer is mixed with a second organic semiconductor, the second organic semiconductor being an N type semiconductor or a P type semiconductor.

5. The carbon nanotube organic semiconductor as claimed in claim 4, wherein at a mixed volume of the carbon nanotube wrapped with the conjugated polymer and the second organic semiconductor, the carbon nanotube wrapped with the conjugated polymer is 10 vol. % or greater in volume.

6. The carbon nanotube organic semiconductor as claimed in claim 4, wherein the N type organic semiconductor is selected from a substance based on acene, fully fluorinated acene, partially fluorinated acene, partially fluorinated oligothiophene, fullerene, fullerne with a substituent, fully fluorinated phthalocyanine, partially fluorinated phthalocyanine, perylene tetracarboxylic diimide, perylene tetracarboxylic dianhydride, naphthalene tetracarboxylic diimide, or naphthalene tetracarboxylic dianhydride, or a derivative thereof,
wherein the P type organic semiconductor is selected from a substance including acene, poly-thienylene vinylene, poly-3-hexylthiophene, alpha-hexathienylene, naphthalene, alpha-6-thiophene, alpha-4-thiophene, rubrene, polythiophene, polyparaphenylene vinylene, polyparaphenylene, polyfluorene, polythiophene vinylene, polythiophene-heterocyclic aromatic copolymer, or triaryl amine, or a derivative thereof.

7. A method for manufacturing a carbon nanotube organic semiconductor, which is a method for manufacturing a layer constituting an organic thin film transistor, the method comprising:
mixing a conjugated polymer and a single-walled carbon nanotube in a solvent;
performing an ultrasonication on the mixed solution;
performing a separation using a centrifugal separator to collet a supernate; and
using the supernate to form a carbon nanotube organic semiconductor forming an organic semiconductor layer,
wherein the supernate in the separation step comprises a single-walled carbon nanotube having semiconducting properties wrapped with a conjugated polymer.

8. The method as claimed in claim 7, wherein the mixing step (1) uses 4 to 6 mg of the conjugated polymer and 1.5 to 3.0 mg of the single-walled carbon nanotube per 1 ml of the solvent,
wherein the mixing ratio of the conjugated polymer to the single-walled carbon nanotube is 3:2 to 3:1.

9. The method as claimed in claim 7, wherein the conjugated polymer is any one selected from the group consisting of polyfluorene, polythiophene, 1,4-diketopyrrolo[3,4-c]pyrrole (DPP), naphthalene diimide, naphthalene-bis(dicarboximide) (NDI), isoindigo, and isothiophene indigo.

10. The method as claimed in claim 7, wherein the solvent is any one selected from the group consisting of toluene, chloroform, chlorobenzene, dichlorobenzene, trichlorobenzene, and xylene.

11. A transistor for chemical sensor comprising:
a substrate;
source/drain electrodes disposed apart from each other on the substrate;
a carbon nanotube organic semiconductor layer comprising a substance formed of a single-walled carbon nanotube having semiconducting properties wrapped with a conjugated polymer and being disposed on the whole surface of the substrate including the source/drain electrodes;
a gate insulating layer being disposed on the whole surface of the organic semiconductor layer; and
a gate electrode being disposed on the gate insulating layer.

12. The transistor for chemical sensor as claimed in claim 11, wherein the conjugated polymer of the carbon nanotube organic semiconductor is any one selected from the group consisting of polyfluorene, polythiophene, 1,4-diketopyrrolo[3,4-c]pyrrole (DPP), naphthalene diimide, naphthalene-bis(dicarboximide) (NDI), isoindigo, and isothiophene indigo.

13. The transistor for chemical sensor as claimed in claim 11, wherein the carbon nanotube organic semiconductor comprises 0.0001 to 0.015 mg/ml of the single-walled carbon nanotube.

14. The transistor for chemical sensor as claimed in claim 11, wherein the organic semiconductor layer uses a second organic semiconductor further added, the second organic semiconductor being an N type organic semiconductor or a P type organic semiconductor.

15. The transistor for chemical sensor as claimed in claim 14, wherein at a mixed volume of the carbon nanotube wrapped with the conjugated polymer and the second organic semiconductor, the carbon nanotube wrapped with the conjugated polymer is 10 vol. % or greater in volume.

16. The transistor for chemical sensor as claimed in claim 11, wherein the transistor is used as an active layer to sense the change of chemical properties upon exposure to a chemical substance and applicable to diagnosis of lung cancer with exhaled breath.

17. The transistor for chemical sensor as claimed in claim 12, wherein the transistor is used as an active layer to sense the change of chemical properties upon exposure to a chemical substance and applicable to diagnosis of lung cancer with exhaled breath.

18. The transistor for chemical sensor as claimed in claim 13, wherein the transistor is used as an active layer to sense the change of chemical properties upon exposure to a chemical substance and applicable to diagnosis of lung cancer with exhaled breath.

19. The transistor for chemical sensor as claimed in claim 14, wherein the transistor is used as an active layer to sense the change of chemical properties upon exposure to a chemical substance and applicable to diagnosis of lung cancer with exhaled breath.

20. The transistor for chemical sensor as claimed in claim 15, wherein the transistor is used as an active layer to sense the change of chemical properties upon exposure to a chemical substance and applicable to diagnosis of lung cancer with exhaled breath.
